(19) United States
(12) Patent Application Publication
(10) Pub. No.: US 2017/0347966 A1
(43) Pub. Date: Dec. 7, 2017

(54) VASCULAR TREATMENT EVALUATION SYSTEM, AND METHOD THEREFOR

(71) Applicant: EBM CORPORATION, Tokyo (JP)

(72) Inventors: Takanobu YAGI, Tokyo (JP); Young-Kwang PARK, Tokyo (JP)

(21) Appl. No.: 15/503,626

(22) PCT Filed: Oct. 8, 2015
(86) PCT No.: PCT/JP2015/078676

§ 371 Date: Jul. 17, 2017

Related U.S. Application Data
(60) Provisional application No. 62/061,409, filed on Oct. 8, 2014.

(51) Int. Cl. A61B 5/00; G06T 7/00; A61B 5/026; G06K 9/62; G06T 7/62; G06T 7/246; G06T 7/50; G06T 15/08

(52) U.S. Cl. CPC A61B 5/7275; A61B 5/026; A61B 5/4848; G06T 7/0012; G06T 15/08; G06T 7/62; A61B 5/004; G06T 7/246; G06T 7/50; G06T 2207/10088; G06T 2207/30104; G06T 2210/41; G06K 9/6267

(57) ABSTRACT

Provided is a system for supporting the treatment of vascular diseases by performing a blood flow simulation based on a medical image, the system comprising: an input unit that reads the medical image, fluid properties and boundary conditions from a data storage unit; a blood flow analysis execution unit that obtains a pressure field and a flow velocity field based on the medical image read by the input unit; a blood flow information calculation unit that calculates, based on the pressure field and the flow velocity field, blood flow information about a specific blood vessel being treated; a vascular treatment risk assessment unit that calculates, based on the calculated blood flow information, the proportion of a blood flow volume flowing into an aneurysm as a risk factor associated with the vascular treatment of the blood vessel being treated; and display units that display the calculation results to a user.

### BACKGROUND OF THE INVENTION

### Field of the Invention

The present invention relates to a system for evaluating vascular treatment risks, and a computer software program and method therefor and particularly to a system for evaluating vascular rupture risks when coil embolization treatment, one of treatment methods for cerebral aneurysms, is performed or evaluating postoperative quality, and a computer software program and method therefor.

Coil embolization treatment has conventionally been performed as one of methods for treating cerebral aneurysms. The coil embolization treatment is a method for lowering a blood flow to make thrombosing occur within an aneurysm by filling the aneurysm with a plurality of coils made of platinum or the like. This is a method for making thrombi in the entire region of the aneurysm, so that a blood flow into the aneurysm is obstructed to prevent aneurysmal rupture.

The coil embolization treatment for cerebral aneurysm has conventionally been performed on the basis of rules of thumb. In other words, the aneurysmal volume, the neck length, the filling rate, etc. have been considered as risk factors at the time of assessing aneurysmal rupture risks, the necessity of treatment, the quantity of coils, etc.

In assessing the quantity of coils to be filled, it is believed that the volume of coils to be filled should be 20-30% relative to the volume of an aneurysm. However, when the aneurysm is large, the neck of the aneurysm is wide or the coil filling rate topically declines due to technical restriction (remaining regions), thrombosing tends to be insufficient, because coils are compressed toward the parietal region of an aneurysm (coil compaction) or the remaining region regrows (neck remnant growth).

Furthermore, even when the quality of treatment is postoperatively considered, the quality of surgery cannot be determined at the non-thrombosed stage immediately after coil embolization treatment, because the thrombosing of an aneurysm does not occur immediately after surgery; therefore the quality of treatment has been determined by observing conditions after surgery. Accordingly, it requires at least several months in order to observe postoperative conditions and determine the quality of treatment.

In all of those cases, as seen in FIG. 1a and FIG. 1b, a blood flow is sometimes reopened, and in such a case additional surgery is considered because the aneurysmal rupture risk increases. Such a case is not rare; it has been reported that such a case occurs in about 20% of all the operations, though there are some differences among facilities. Furthermore, in the case of small aneurysms, it becomes difficult to perform coil embolization treatment and any expected filling rate might not be achieved. In the case of giant aneurysms, the problem is that an extremely large amount of coils is required in order to attain an expected filling rate, which leads to an increase in medical expenses.

### SUMMARY OF THE INVENTION

In the conventional technology relating to coil embolization treatment as described above in the section of the background of the invention, doctors has been making a determination after all on the basis of the shape elements of aneurysms in all cases, which is based on rules of thumb.

The present invention was made in view of the abovementioned problems; the present invention is to provide a system for lowering risks associated with coil embolization treatment, that is, a system for supporting treatment in assessing aneurysmal rupture risks, the necessity of treatment, the quantity of coils, etc., and a computer software program and method therefor in order to address the abovementioned problems.

In a first major point of this invention, provided is a system for evaluating vascular treatment on the basis of a medical image, the system comprising: a blood flow information calculation unit that calculates blood flow information about a specific blood vessel being treated; a vascular treatment risk assessment unit that calculates, on the basis of the calculated blood flow information, a risk factor associated with the vascular treatment of the blood vessel being treated; and a display unit that displays the calculated risk factor to a user.

Such a configuration makes it possible to calculate a risk factor associated with coil embolization treatment of an aneurysm, for example, on the basis of blood flow information about a blood flow volume obtained from a medical image and then provide it to a user.

In one embodiment of this invention, this system has the blood flow information calculation unit calculate a blood flow volume flowing into the specific blood vessel being treated as a risk factor. In the case that the specific blood vessel being treated is an aneurysm, the blood flow information calculation unit preferably calculates the proportion of a blood flow volume flowing into the aneurysm from a parent blood vessel as a risk factor prior to the vascular treatment. In this case, it is further desirable that the vascular treatment risk assessment unit assess, on the basis of the proportion of the blood flow volume flowing into the aneurysm as the risk factor, the growth risk/rupture risk of this aneurysm prior to the treatment. In this case, the assessment of the growth risk/rupture risk is preferably made on the basis of the classification of the proportion of blood flow volumes flowing into aneurysms that grew or did not grow in the past.

In another embodiment of this invention, the vascular treatment risk assessment unit calculates the ratio between a blood flow volume flowing into the aneurysm from a parent blood vessel prior to the vascular treatment and a blood flow volume flowing into the aneurysm after the vascular treatment as a risk factor showing the quality of the vascular treatment. The assessment of the quality of vascular treatment is preferably made on the basis of the classification of the flow rate proportion in cases where retreatment was required after treatment and the flow rate proportion in cases where retreatment was not required. Furthermore, in this case, it is desirable to be assessed that the result of vascular treatment is not good when the flow rate proportion of posttreatment to pretreatment is 200% or more.

In another embodiment of this invention, the vascular treatment is coil embolization treatment for an aneurysm. However, the present invention is not limited to this example but may be used for a clipping method and a balloon/stent filling method as well.

In a second major point of this invention, provided is a computer software program for supporting the treatment of vascular diseases by performing a blood flow simulation on the basis of a medical image, the program executing the abovementioned system that comprises: a step for having a computer calculate blood flow information about a specific blood vessel being treated; a step for calculating, on the basis of the calculated blood flow information, a risk factor associated with the vascular treatment of the blood vessel being treated; and a step for displaying the calculation result to a user.

Furthermore, in a third major point of this invention, provided is a method for supporting the treatment of vascular diseases by performing a blood flow simulation on the basis of a medical image, the method comprising: a step for calculating blood flow information about a specific blood vessel being treated; a step for calculating, on the basis of the calculated blood flow information, a risk factor associated with the vascular treatment of the blood vessel being treated; and a step for displaying the calculated result to a user.

The characteristics of the present invention that are not described above will be made clear in the section of the detailed description of the invention below as well as by drawings in such a way as to be enforceable by those skilled in the art.

### BRIEF DESCRIPTION OF THE DRAWINGS

FIG. 1a is a view showing the state of an aneurysm immediately after performing coil embolization treatment.

FIG. 1b is a view showing the state of the aneurysm one year after performing coil embolization treatment.

FIG. 2 is a schematic system block diagram showing one embodiment of the present invention.

FIG. 3 is a flow diagram showing the construction of a flow passage shape according to one embodiment.

FIG. 4 is a flow diagram showing blood flow analysis according to one embodiment.

FIG. 5 is a view showing a blood flow volume in a blood vessel and an aneurysm using flow lines.

FIG. 6 is a reference view for defining the neck face of a cerebral aneurysm.

FIG. 7a is a view showing a remaining region in an aneurysm immediately after performing coil embolization treatment.

FIG. 7b is a view showing a reopened region in the aneurysm one year after performing coil embolization treatment.

FIG. 8 is a view showing the inflow coefficient according to one embodiment of the present invention.

FIG. 9 is a table showing the relationship between the inflow coefficient and grades.

FIG. 10 is a view showing data relative to the volume of aneurysms.

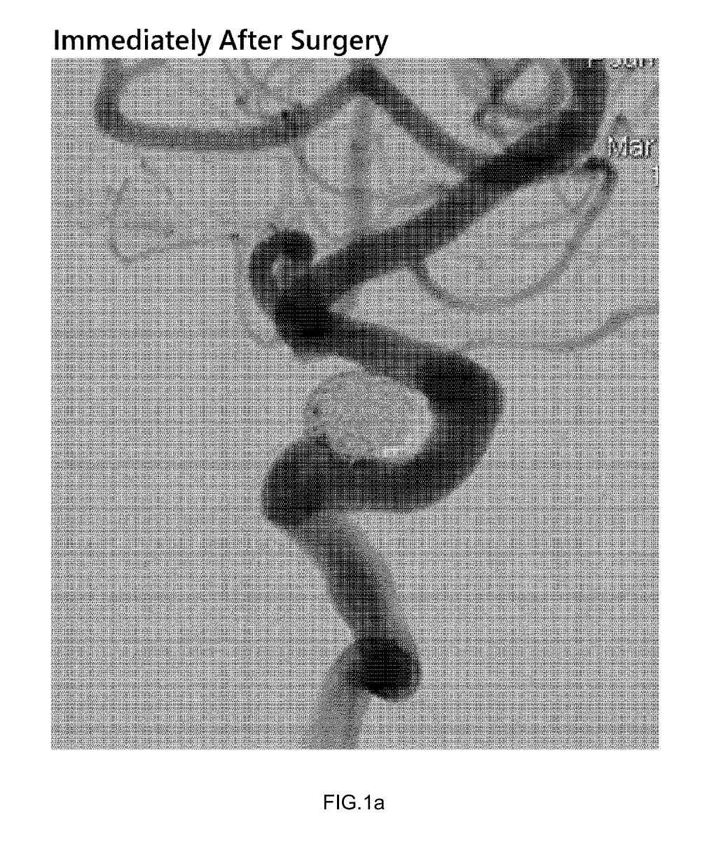

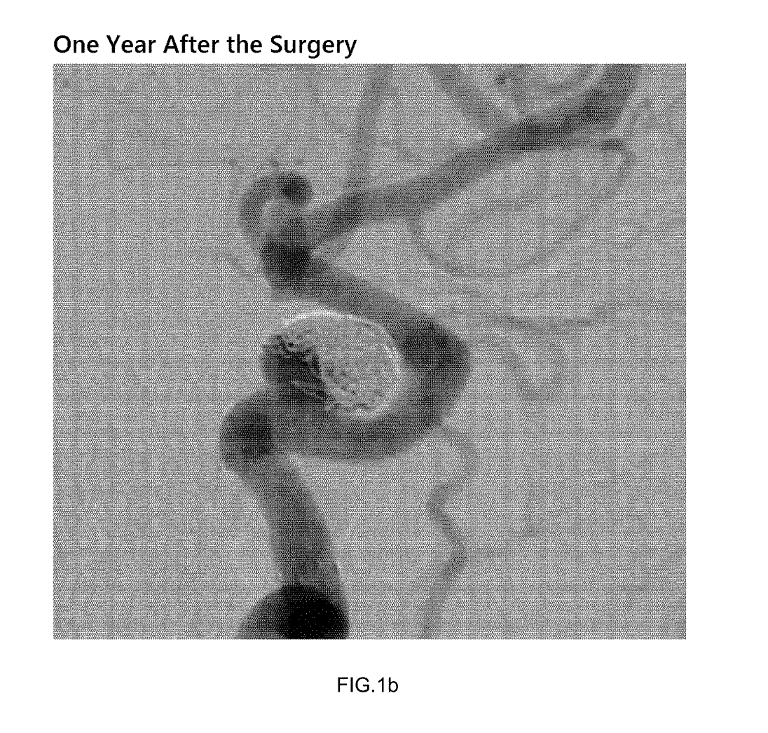

### DETAILED DESCRIPTION OF THE INVENTION

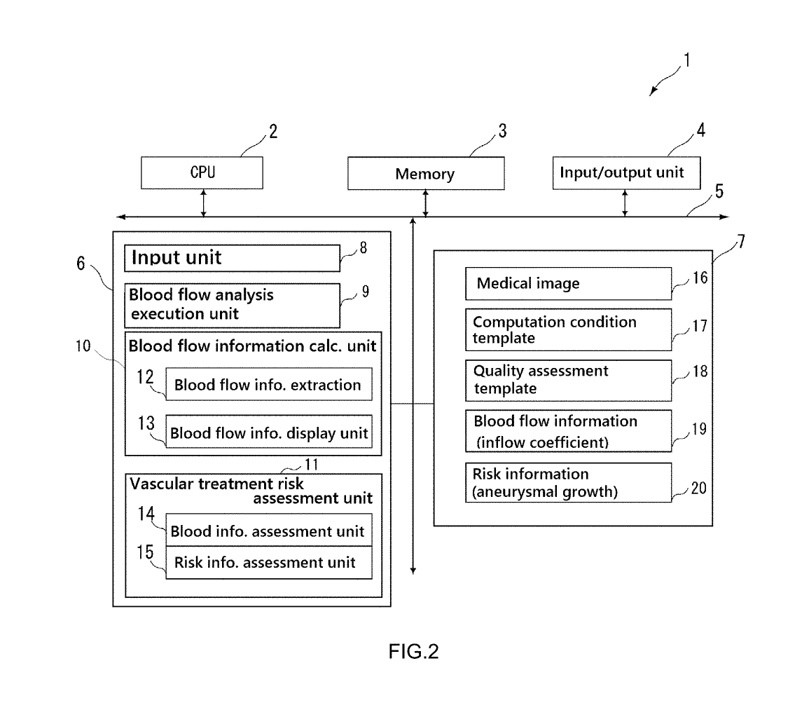

A description of one preferable embodiment of the present invention is given below in detail with reference to FIGS. 2-10.

### Overall Structure

FIG. 2 is a schematic system block diagram of a blood flow analyzer 1. This blood flow analyzer 1 is composed of a program storage unit 6 and a data storage unit 7 that are connected to a bus 5 to which a CPU 2, a memory 3 and an input/output unit 4 are connected. The program storage unit 6 is provided with an input unit 8, a blood flow analysis execution unit 9, a blood flow information calculation unit 10 and a vascular treatment risk assessment unit 11. The blood flow information calculation unit 10 comprises a blood flow information extraction unit 12 and a blood flow information display unit 13, and the vascular treatment risk assessment unit 11 comprises a blood flow information assessment unit 14 and a risk information display unit 15. The data storage unit 7 stores a medical image 16, a computation condition template 17, a quality assessment template 18, blood flow information 19 and risk information.

The constituent elements (8-20) are actually composed of computer software stored in the storage space of a hard disk and retrieved by the CPU 2 to be developed and executed on the memory 3; each constituent element of this invention is constituted and functioned in this manner.

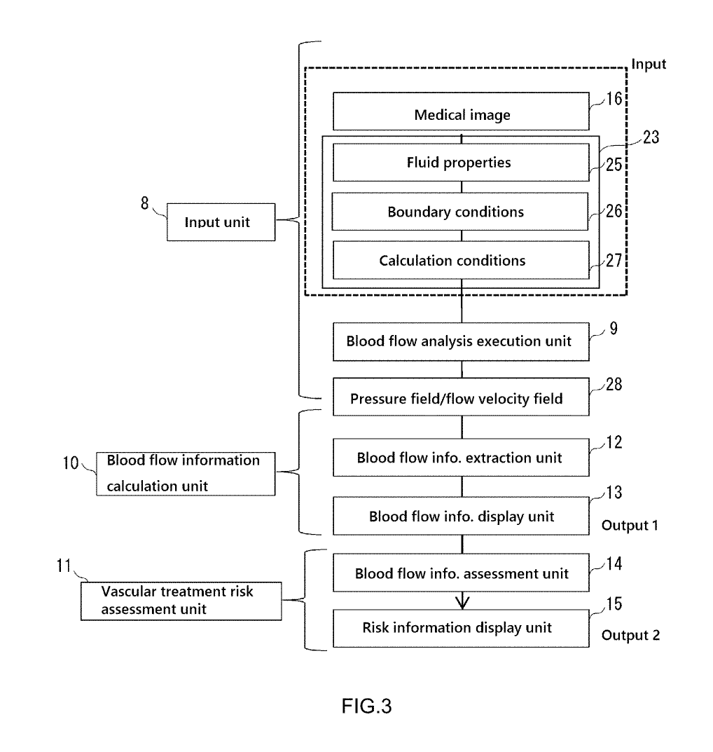

The following describes the configuration of each constituent element (8-20) of the abovementioned blood flow analyzer 1 in detail with reference to a flow diagram shown in FIG. 3.

### Input Unit

The input unit 8 receives the medical image 16, the fluid properties 25, the boundary conditions 26 and the calculation conditions 27 from the data storage unit 7. The medical image 16 is an MM image or the like. The Fluid properties 25 are density and viscosity in this embodiment. The boundary conditions 26 are a flow velocity, a pressure distribution and restriction conditions at the wall face of each conduit. In this embodiment, the velocity is set to zero by disregarding the flow velocity distribution at inlets and outlets and the slip of fluid at the wall face (non-slip condition). The calculation conditions 27 are to generate a computational mesh for a given flow passage shape and is the discretization of equations for equation solving and a solution of simultaneous equations.

### Blood Flow Analysis Execution Unit

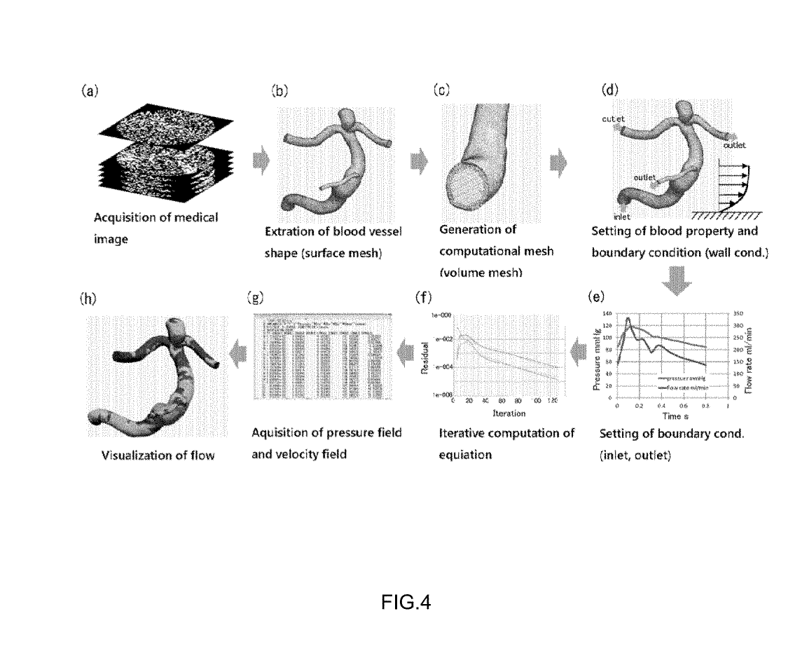

The blood flow analysis execution unit 9 obtains, on the basis of the medical image 16 read by the input unit 8, a pressure field and a flow velocity field. As shown in FIG. 4, the blood flow analysis execution unit 9 first receives the medical image 16 (a). Next, it extracts a blood vessel shape (surface mesh) on the basis of the received medical image (b), generates calculation meshes (volume mesh) (c), sets the fluid properties 25 and the boundary conditions (wall face) 26 inputted by the input unit 8 (d) and then sets a flow rate and a flow pressure at the inlet and outlet of the blood flow (e). By calculating an equation iteratively on the basis of the flow rate and pressure that have been set (f), the pressure field and flow velocity field are obtained; this pressure field and flow velocity field will be the pressure field and flow velocity field in the time and space when solving them as a time development type.

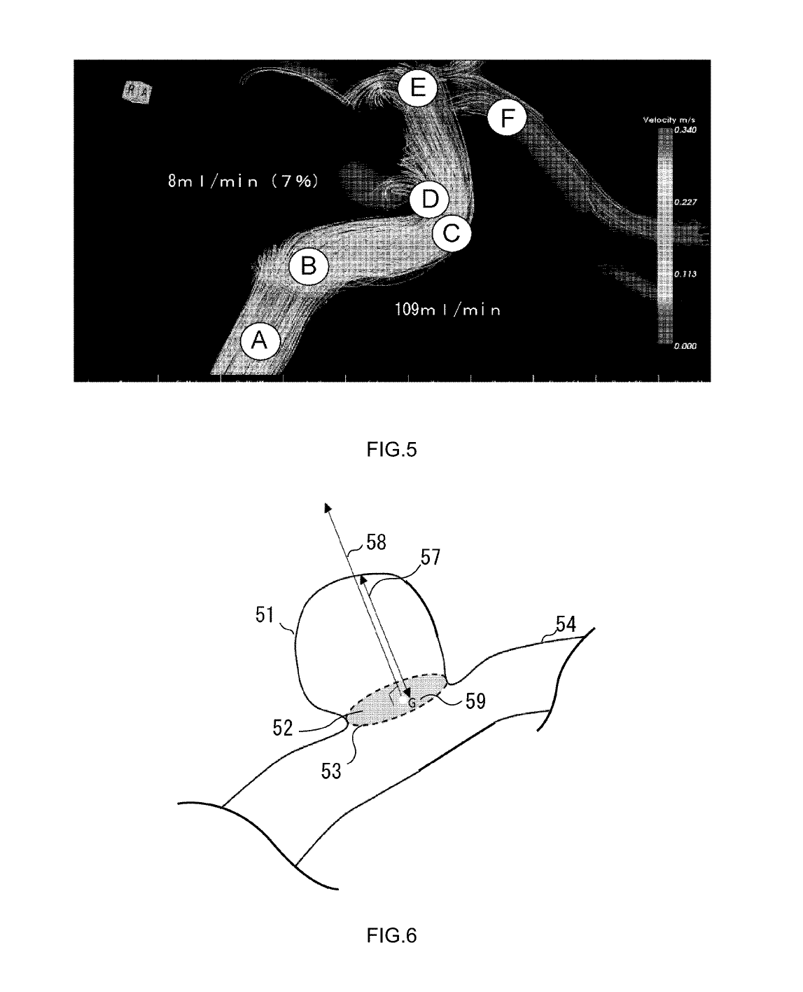

FIG. 5 is a view showing the flow line of a blood flow using visualization on the basis of the obtained pressure field and flow velocity field, wherein the level of flow velocity is represented in colors. By way of example, a blood flow at a low flow velocity is represented in blue; colors are gradually changed by gradation to light blue, green, yellow, orange, etc.; and a blood flow at a high low velocity is represented in red. For example, as shown in FIG. 5, the region represented by A is drawn with green, yellow and red lines; B is shown in light blue and green; C is drawn approximately with a green line; D is shown in red and yellow: and E and F are approximately constituted of red lines. Thus, the flow velocity is visualized in colors. Furthermore, D is located in the vicinity of the inlet of the aneurysm; the extension of flow lines into the aneurysm shows that blood is flowing into the aneurysm.

### Blood Flow Information Calculation Unit

The blood flow information calculation unit 10 calculates, on the basis of the abovementioned pressure field and flow velocity field 28 found by the blood flow analysis execution unit 9, a blood flow volume, which is one of state quantities within an aneurysm, that is, an inflow coefficient. FIG. 6 is a schematic view explaining the calculation of this inflow coefficient. In FIG. 6, a reference numeral 51 is an aneurysm, and a reference numeral 54 is a blood vessel. A plane located at a neck region 53 of the aneurysm 51, which is the boundary between 54 and 51, is referred to as a neck face 52.

In this embodiment, the center G 59 of the neck face 52 is first determined, and then a unit vector 57 in the vertical direction within the aneurysm, which is oriented toward the vertical direction 58 from the center, is extracted. The velocity of blood substantially flowing into the aneurysm is calculated by finding the inner product of the velocity vector within the neck face 52, which is calculated on the basis of the unit vector 58 in the vertical direction within the aneurysm and the abovementioned pressure field and flow velocity field. This velocity becomes zero if the entire face is integrated; this is because the blood inflow volume is equal to the blood outflow volume. Accordingly, either one of the blood inflow volume or the blood outflow volume may be referenced; however, only the blood inflow volume is referenced here. Given that a flow line moving toward the vertical direction within the aneurysm is positive, the blood inflow volume can be calculated by adding only positive flow volumes. By way of example, in the example shown in FIG. 5, it is 8 mL/min. If it is divided by the flow volume of the parent blood vessel (109 mL/min), the inflow coefficient is 0.07 (i.e., 7%), that is, it is shown that 7% of the flow volume of the parent blood vessel is flowing into the aneurysm.

### Vascular Treatment Risk Assessment Unit

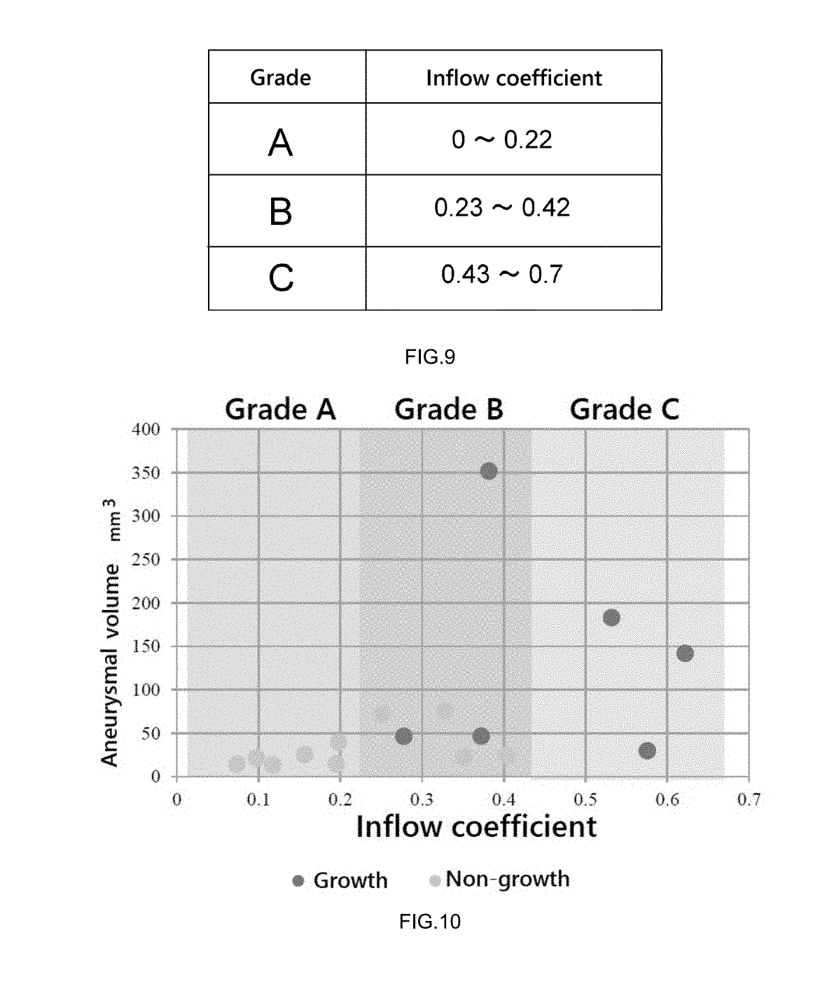

The vascular treatment risk assessment unit 11 reads the quality assessment template 18 stored in the data storage unit 7 and checks the inflow coefficient of blood flowing into the aneurysm, which is calculated by the abovementioned blood flow information calculation unit 10, against the quality assessment template 18 to assess aneurysmal growth or the possibility (risk) of additional surgery. In this embodiment, as shown in FIG. 9, the vascular treatment risk assessment unit assesses Grade A when the inflow coefficient is 0-0.22, Grade B when it is 0.23-0.42 and Grade C when it is 0.43-0.7. This assessment is determined on the basis of accumulated data about the growth and non-growth of aneurysms; in the present example, as shown in FIG. 10, Grade A is set when the inflow coefficient is 0-0.22 because there is no case of aneurysmal growth, Grade B is set because cases of aneurysmal growth and non-growth are mixed, and Grade C is set because cases of aneurysmal growth is 100% when the inflow coefficient is 0.43 or above. On the basis of this assessment, the risk information display unit 15 displays evaluation results as follows: there is substantially no risk at Grade A; special attention is required at Grade B; and there is a high risk at Grade C.

Furthermore, FIG. 10 is a view showing data about the inflow coefficient calculated by the blood flow information calculation unit 10 relative to the aneurysmal volume. Cases in which the inflow coefficient increases are sometimes found even when the volume is 50 mm³ or less. In other words, this shows that it is insufficient to use the volume alone as a risk factor. In fact, the inflow coefficient ranges from 0.1 or less (minimum) to approximately 0.6 (maximum) in the same zone. Accordingly, it is demonstrated that fluid characteristics cannot be evaluated by the shape of aneurysms alone and that evaluation using the flow rate counting according to the present invention is effective.

Furthermore, the following explains the evaluation of surgery after performing coil embolization treatment as another example using the inflow coefficient.

In the present example, a template is prepared in advance that stores a numerical value obtained by comparatively examining cases in which additional surgery was required after performing coil embolization treatment and cases in which no additional surgery was required (postoperative flow rate/postoperative flow rate) as a template for postoperatively assessing the quality of surgery, and a computer calculates the quality of surgery after performing coil embolization treatment on the basis of the abovementioned examination. In the present embodiment, this numerical value is set to 200% or higher. This is a numerical value obtained by comparatively examining cases in which additional surgery was required after performing coil embolization treatment and cases in which no additional surgery was required, on the basis of the facts that the relative blood flow volume flowing into cerebral aneurysms approximately doubled in cases in which additional surgery was required and that approximately 50% of blood flowed into aneurysms in those cases in which additional surgery was required.

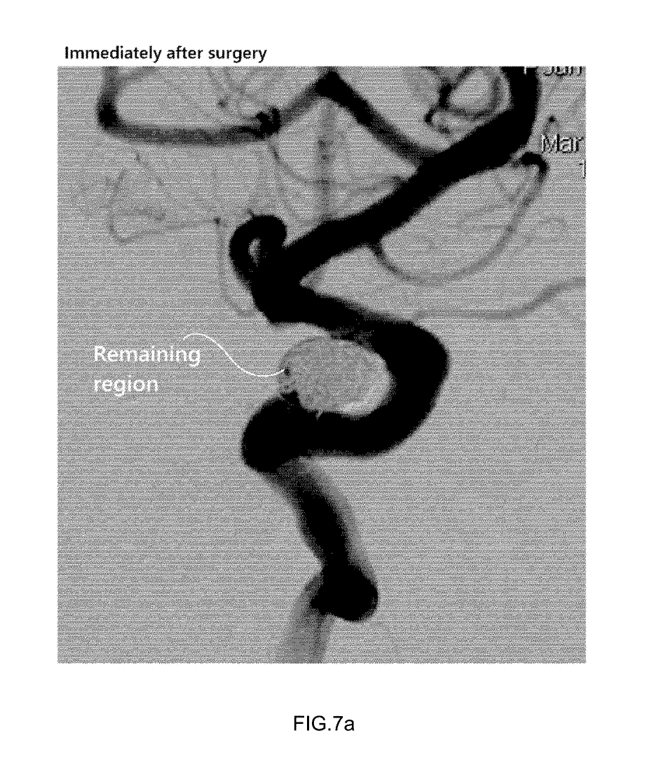

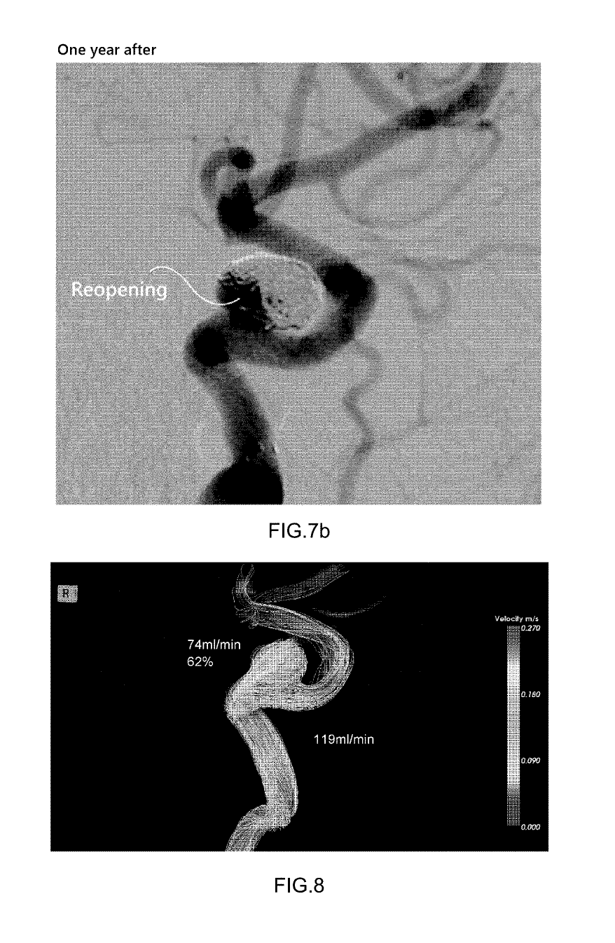

FIG. 7a and FIG. 7b are MRI images of cases in which reopening occurred immediately after performing coil embolization treatment and one year later, respectively. The comparison of the state of the aneurysm immediately after surgery and one year later shows that the blood inflow region of the remaining region agrees with that of the reopened region. The calculation of the volume of inflow blood by the abovementioned blood flow information calculation unit showed that the inflow coefficient reached as high as 62%. In other words, blood of inflow coefficient 62% was flowing into the aneurysm from the same inflow region in this case; therefore, it is demonstrated that the risk of blood flow increase can be found immediately after surgery by examining the inflow coefficient at such an early stage.

In developing the aneurysmal treatment supporting tool according to the present invention, the present inventors paid attention to the following point: thrombi within cerebral aneurysms are related to the blood flow and induced by a decline in the blood flow; and thus this issue should be handled by the blood flow element rather than the shape element. In other words, as described above, because shape elements such as the aneurysmal volume, the neck length and the filling rate are used as risk factors in the conventional way, it does not follow that the blood flow has been evaluated. Instead of the conventional shape elements, the present inventors paid attention to the blood flow volume flowing into cerebral aneurysms and found that the risk of aneurysmal growth and aneurysmal rupture can be predicted by calculating the ratio between the blood flow volume flowing into a parent blood vessel and the blood flow volume flowing into an aneurysm as an aneurysmal inflow coefficient, thereby completed the present invention.

Such a constitution is effective in easily assessing the risk of aneurysmal growth and postoperative reopening on the basis of the inflow coefficient rather than depending on assessment made by doctors on the basis of the shape elements of aneurysms.

In other words, in the study conducted by the inventors, it has been shown that there are cases with cerebral aneurysms in which the blood inflow volume from the parent blood vessel is 10% or so or less than 10%. That is, it has been shown that there are some cerebral aneurysms that are not connected to blood vessels any more in terms of the blood flow after developing the aneurysms, though they are connected to those blood vessels morphologically. The use of the device according to the present invention makes it possible, in addition to lowering risks, to eliminate the necessity to perform coil embolization treatment for aneurysms with no blood inflow or reduce the filling rate, that is, it is possible to lower the cost. As described above, the present invention is effective in supporting coil embolization treatment in many ways.

The abovementioned explanation is only about one example of the present invention and can be modified in various manners without departing from the scope of the invention.

By way of example, in the abovementioned embodiment, blood flow information about vascular treatment is the blood flow volume and risk factors are blood flow volume ratio, etc., but the present invention is not limited to this example. For example, the abovementioned blood flow information may be about flow velocity, energy, pressure or the like as long as it is some type of quantity showing the state of blood flowing into a cerebral aneurysm.

Furthermore, in the abovementioned embodiment, vascular treatment is coil embolization treatment, but the present invention is not limited to this example. It may be a clipping method or a balloon/stent filling method.

1. A system for evaluating vascular treatment based on a medical image, the system comprising:
a blood flow information calculation unit that calculates blood flow information of a specific blood vessel being treated;
a vascular treatment risk assessment unit that calculates, based on the blood flow information, a risk factor associated with vascular treatment of the specific blood vessel being treated; and
a display unit that displays the risk factor to a user.

2. The system according to claim 1, wherein the blood flow information calculation unit calculates a blood flow volume flowing into the specific blood vessel being treated as the risk factor.

3. The system according to claim 2, wherein the specific blood vessel being treated is an aneurysm, and the blood flow information calculation unit calculates a proportion of blood flow volume flowing into the aneurysm from a mainstream of blood vessels as the risk factor in a stage prior to the vascular treatment.

4. The system according to claim 3, wherein the vascular treatment risk assessment unit assesses growth risk/rupture risk of this aneurysm prior to the treatment based on the proportion of the blood flow volume flowing into the aneurysm as the risk factor.

5. The system according to claim 4, wherein the growth risk/rupture risk is assessed based on a classification of a proportion of blood flow volumes flowing into the aneurysms that grew or did not grow in the past.

6. The system according to claim 2, wherein the specific blood vessel being treated is an aneurysm, and the vascular treatment risk assessment unit calculates a proportion of a blood flow volume flowing into the aneurysm from a parent blood vessel prior to the vascular treatment to a blood flow volume flowing into the aneurysm after the vascular treatment as a risk factor showing quality of the vascular treatment.

7. The system according to claim 6, wherein the quality of vascular treatment is assessed based on a classification of a flow rate proportion in cases where retreatment was required after the vascular treatment and the flow rate proportion in cases where the retreatment was not required.

8. The system according to claim 7, wherein the vascular treatment risk assessment unit assesses that the result of vascular treatment is not good when the flow rate proportion of posttreatment to pretreatment is 200% or more.

9. The system according to claim 1, wherein the vascular treatment is coil embolization treatment for an aneurysm.

10-18. (canceled)

19. A method for evaluating vascular treatment based on a medical image, the method comprising:
a step for calculating blood flow information of a specific blood vessel being treated;
a step for calculating a risk factor associated with vascular treatment of the specific blood vessel being treated, based on the blood flow information; and
a step for displaying the risk factor to a user.

20. The method according to claim 19, wherein the step for calculating blood flow information of the specific blood vessel being treated calculates a blood flow volume flowing into the specific blood vessel being treated as the risk factor.

21. The method according to claim 20, wherein the specific blood vessel being treated is an aneurysm, and the step for calculating blood flow information of the specific blood vessel being treated calculates the proportion of a blood flow volume flowing into the aneurysm from the mainstream of blood vessels as a risk factor in a state prior to the vascular treatment.

22. The method according to claim 21, wherein in the step for calculating a risk factor associated with the vascular treatment of the specific blood vessel being treated based on the blood flow information, the growth risk/rupture risk of this aneurysm prior to the treatment based on the proportion of the blood flow volume flowing into the aneurysm as the risk factor.

23. The method according to claim 22, wherein the growth risk/rupture risk is assessed based on a classification of a proportion of blood flow volumes flowing into the aneurysms that grew or did not grow in the past.

24. The method according to claim 20, wherein the specific blood vessel being treated is an aneurysm, and the step for calculating a risk factor associated with the vascular treatment of the specific blood vessel being treated based on the blood flow information calculates the proportion of a blood flow volume flowing into the aneurysm from a parent blood vessel prior to the vascular treatment to a blood flow volume flowing into the aneurysm after the vascular treatment as a risk factor showing quality of the vascular treatment.

25. The method according to claim 24, wherein the quality of vascular treatment is done based on a classification of the flow rate proportion in cases where retreatment was required after the vascular treatment and the flow rate proportion in cases where the retreatment was not required.

26. The method according to claim 25, wherein the step for calculating, based on the blood flow information, a risk factor associated with the vascular treatment of the specific blood vessel being treated assesses that the result of vascular treatment is not good when the flow rate proportion of posttreatment to pretreatment is 200% or more.

27. The method according to claim 19, wherein the vascular treatment is coil embolization treatment for an aneurysm.
